(19) United States
(12) Patent Application Publication
(10) Pub. No.: US 2020/0306497 A1
(43) Pub. Date: Oct. 1, 2020

(54) SLEEP MASK WITH AROMATHERAPY CAPABILITIES

(71) Applicant: Philip Alexander Herrador, Orlando, FL (US)

(72) Inventor: Philip Alexander HERRADOR, Orlando, FL (US)

(21) Appl. No.: 16/835,406

(22) Filed: Mar. 31, 2020

Related U.S. Application Data
(60) Provisional application No. 62/827,154, filed on Mar. 31, 2019.

(51) Int. Cl. A61M 21/02; A61F 9/04

(52) U.S. Cl. CPC A61M 21/02; A61M 2021/0016; A61F 9/04

(57) ABSTRACT

A sleep mask including a front side, wherein the front side is a layered fabric, a back side, wherein the back side is a layer fabric, a top, a bottom, a left side, a right side, an adjustable strap, the strap connected to the left and right sides, eye pockets, wherein the eye pockets are located on the back side, a nasal arch having a top, a first side, and a second side, wherein the nasal arch is located substantially in the center of the bottom, a light shield, the light shield attached around the nasal arch on the back side, and two diffuser holders fixedly attached to the first and second sides of the nasal arch, wherein each diffuser holder has an interior cavity and an opening, wherein the opening is smaller than the interior cavity, wherein the interior cavity is adapted to receive diffuser material.

This application claims priority to U.S. Ser. No. 62/827,154, filed Mar. 31, 2019, entitled Sleep Mask With Aromatherapy Capabilities, the entirety of which is incorporated herein by reference.

### I. BACKGROUND

The present teachings relate to a sleep mask with an attached aromatherapy diffuser. More particularly, a mask worn over the eyes that can be used portably during any time of rest, and has the ability to hold different aromatherapy oils interchangeably using an attached diffuser holder.

The present teachings generally relate to both sleep masks and aromatherapy, as examples only. It is to be understood that the present teachings can be used with any device chosen using sound engineering judgment. Sleep masks are used by many individuals, both at home and during times of travel. These masks can be worn over the user's eyes and extend downwardly over the face to provide a darkened environment, which is most optimal for rest and relaxation. Aromatherapy is a rapidly growing area of interest for many essential oil enthusiasts. Aromatherapy is the aromatic use of essential oils for their therapeutic benefits, which include physical and mental wellbeing. The positive benefits of both sleep masks and aromatherapy create the need for a product that combines both.

### II. SUMMARY

The present teachings use a sleep mask with attached aromatherapy diffuser holders for use with essential oils. The mask can be made from various suitable fabrics (with some non-limiting examples being cotton, felt, polyester, and silk) layered together to create a comfortable light blocking mask. An adjustable or fixed size strap centrally attached to the left and right side of the mask can be used to secure it over the user's eyes. The mask will also have one or more holders for an oil diffusing material (with some non-limiting examples being wool, cotton, lava stone, and wood). These holders will be located on the lower half of the mask near the nasal arch, and can be made from any material that will not deteriorate upon contact with the oils (with one non-limiting example being silicone). The diffuser material can be easily removed and replaced as desired to meet the user's aromatherapy scent needs.

Other benefits and advantages will become apparent to those skilled in the art to which it pertains upon reading and understanding of the following detailed specification.

### III. FIGURES

The present teachings are described hereinafter with reference to the accompanying drawings.

FIG. 1 is a front view of the mask, with two diffusers;

FIG. 1A is a cutaway view of the mask, showing the diffusers with stones;

FIG. 2 is a rear view of the mask showing the eye pocket and nasal arch light blocking shield;

FIG. 3 is a rear view of the mask showing a securing mechanism;

FIG. 3A is a rear view of the mask showing a securing mechanism; and

FIG. 4 is a perspective view of the mask on a user's head.

### IV. DETAILED DESCRIPTION

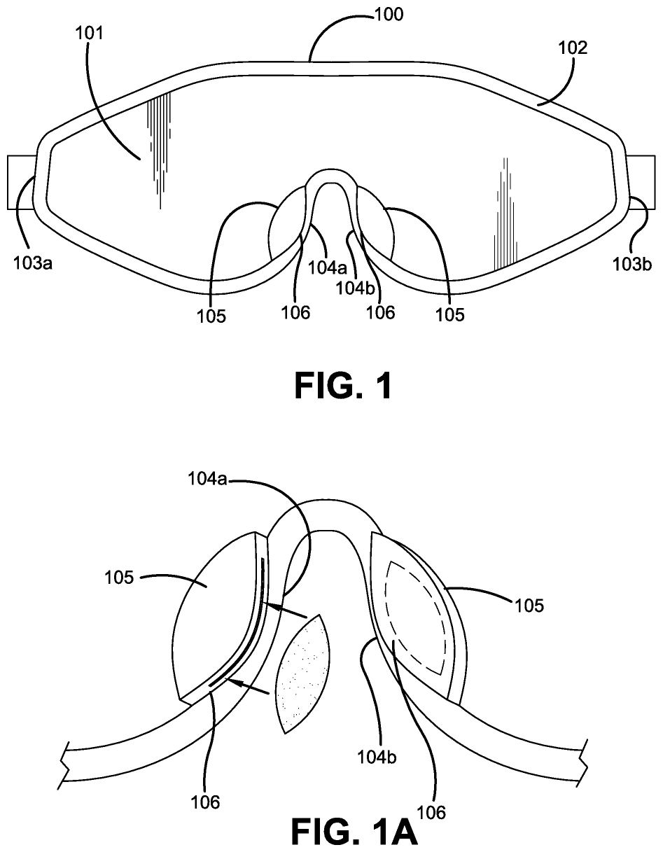

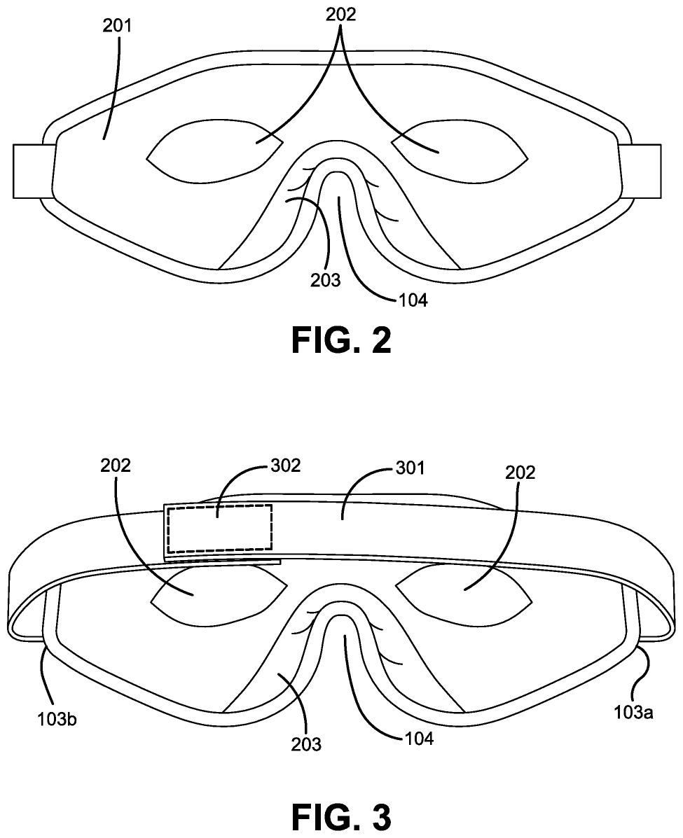

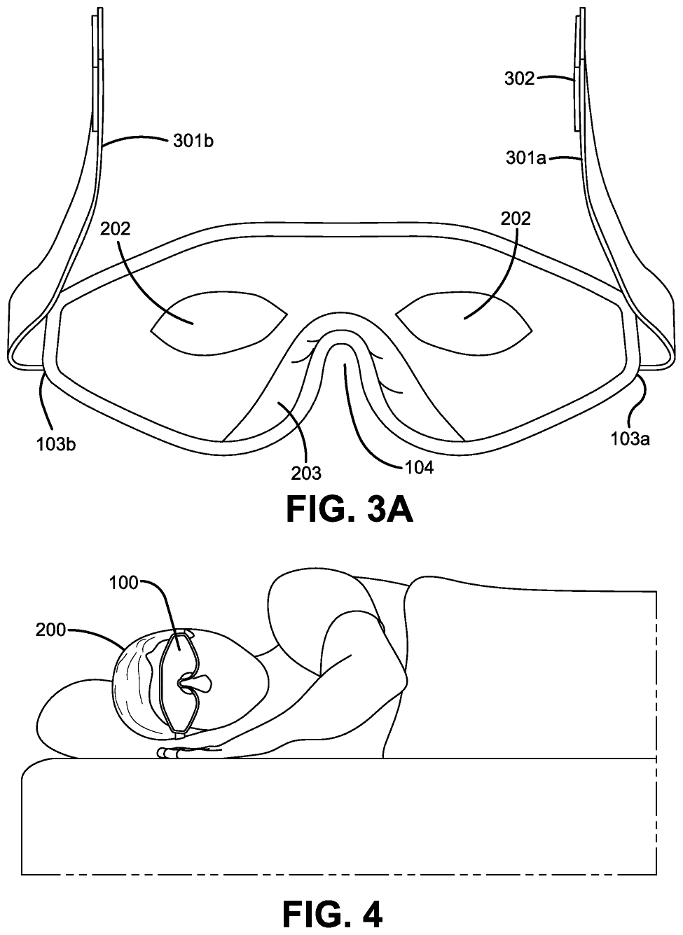

FIGS. 1-4 show a mask 100 with layered fabric 101 shaped to fit a user's head 200, over the user's eyes, and extend downwardly with a contour located near a nasal arch 104. The layered fabric 101 has a stitched trim 102 that wraps along the outer edge of the mask 100 and prevents the edges of the layered fabric 101 from separating. An adjustable elastic strap 301 shown in FIG. 3 is centrally attached to the left and right sides 103a, 103b of the mask 100. This strap 301 can be adjusted in size to be secured around the user's head 200 using an adjustable hook and loop tab 302 as seen in FIG. 3. In this aspect of the present teachings, the strap has a first end 301a and a second end 301b, which are connected together with the adjustable hook and loop tab 302. It is to be understood that any means of attaching the mask 100 to the user's head 200 can be used, as long as chosen using sound engineering judgment. The strap 301 could be all one piece, made of as stretchable material, such that the mask 100 could utilized on various size and shape of user's head 200. The mask 100 could also be attached to a user's head 200 in ways other than the strap 301 described herein, as long as the mask 100 can be secured to the user's head 200.

With continuing reference to FIGS. 1-4, diffuser holders 105 are located on each side 104a, 104b of the nasal arch 104. The diffuser holders 105 can be made of a durable silicone and feature a hollow cavity 106 that has the ability to hold any appropriately sized diffuser pad or lava stone (shown but not referenced). An opening in the diffuser holders 105 is slightly smaller than the cavity 106 to securely hold the diffuser material, and prevent the diffuser material from falling out of the diffuser holders 105. The diffuser holders 105 are either fixedly or removable attached to the mask 100, and can be attached in any manner chosen using sound engineering judgment. Although in one aspect of the present teaching, the diffuser holders 105 are made of silicone, the diffuser holder 105 can be made of any material sufficiently designed to hold the diffuser material. The diffuser material (lava stone, pad, etc.) can be inserted and removed from the hollow cavity 106 in the diffuser holder 105 as often as desired by the user. Although in this aspect of the present teachings, the diffuser holders are located on the sides 104a, 104b of the nasal arch 104, it is to be understood that there could be only one diffuser holder 105, or more than two diffuser holders 105, and that the diffuser holders 105 could be located anywhere on the mask 100, as long as the user can detect the diffuser material. In another aspect of the present teachings, the diffuser holders 105 could be flaps sewn into the mask 100, wherein the diffuser holders 105 would still have cavity 106 for holding the diffuser material. The flaps could be selectively openable such that the diffuser material could be held inside the cavity 106.

With continuing reference to FIGS. 1-4, the reverse side of the mask 100 can be made from a smooth satin or silk fabric layer 201. The reverse side has recessed eye pockets 202 centrally located on the left and right sides 103a, 103b of the mask 100. The eye pockets 202 can be deep enough to not apply pressure to the user's eyes when worn. A light blocking shield 203 is attached just above the nasal arch 104. The shield 203 follows the trim 102, starting from the bottom center of the left side 103a of the mask 100, around the top of the nasal arch 104, and concluding at the bottom center of the right side 103b of the mask 100. The layered fabric 101 and fabric layer 201 can be made of any material chosen using sound engineering judgment, that would be flexible and comfortable for the user. The shield 203 is made of flexible material such that when on the user's head, the shield 203 conforms to the shape of the user's nose, thereby preventing light from seeping under the nasal arch 104 of the mask 100.

Clause 1—A sleep mask comprising a front side, wherein the front side is a layered fabric, a back side, wherein the back side is a layer fabric, a top, a bottom, a left side, a right side, an adjustable strap, the strap connected to the left and right sides, eye pockets, wherein the eye pockets are located on the back side, a nasal arch having a top, a first side, and a second side, wherein the nasal arch is located substantially in the center of the bottom, a light shield, the light shield attached around the nasal arch on the back side, and two diffuser holders fixedly attached to the first and second sides of the nasal arch, wherein each diffuser holder has an interior cavity and an opening, wherein the opening is smaller than the interior cavity, wherein the interior cavity is adapted to receive diffuser material.

Clause 2—A mask comprising a front side, a back side, a top, a bottom, a left side, a right side, and at least one diffuser holder, wherein the at least one diffuser holder has an interior cavity.

Clause 3—The mask of clause 2, wherein the mask further comprises an adjustable strap, wherein the strap is connected to the left and right sides.

Clause 4—The mask of clauses 2 or 3, wherein the mask further comprises a nasal arch having a top, a first side, and a second side, wherein the nasal arch is located substantially in the center of the bottom.

Clause 5—The mask of clauses 2-4, wherein the mask further comprises a light shield, the light shield attached around the nasal arch on the back side.

Clause 6—The mask of clauses 2-5, wherein the mask further comprises eye pockets, wherein the eye pockets are located on the back side.

Clause 7—The mask of clauses 2-6, wherein the at least one diffuser holder is at least two diffuser holders fixedly attached to the first and second sides of the nasal arch, wherein each diffuser holder has an opening, wherein the opening is smaller than the interior cavity, wherein the interior cavity is adapted to receive diffuser material.

Clause 8—The mask of clauses 2-7, wherein the front side and the back side are made of layered fabric.

Clause 9—The mask of clauses 2-8, wherein the layered fabric is silk or satin.

Clause 10—A method for using a sleep mask for aromatherapy, the method comprising the steps of providing a sleep mask comprising a front side, a back side, a top, a bottom, a left side, a right side, and at least one diffuser holder, wherein the at least one diffuser holder has an interior cavity, inserting diffuser material into the at least one diffuser holder, and applying the sleep mask to a user's head.

The various aspects have been described, hereinabove. It will be apparent to those skilled in the art that the above methods and apparatuses may incorporate changes and modifications without departing from the general scope of the present teachings. It is intended to include all such modifications and alterations insofar as they come within the scope of the appended claims or the equivalents thereof. Although the description above contains much specificity, this should not be construed as limiting the scope of the present teachings, but as merely providing illustrations of some of the aspects of the present teachings. Various other aspects and ramifications are possible within its scope.

Furthermore, notwithstanding that the numerical ranges and parameters setting forth the broad scope of the present teachings are approximations, the numerical values set forth in the specific examples are reported as precisely as possible. Any numerical value, however, inherently contain certain errors necessarily resulting from the standard deviation found in their respective testing measurements.

1. A sleep mask comprising:
a front side, wherein the front side is a layered fabric;
a back side, wherein the back side is a layer fabric;
a top;
a bottom;
a left side;
a right side;
an adjustable strap, the strap connected to the left and right sides;
eye pockets, wherein the eye pockets are located on the back side;
a nasal arch having a top, a first side, and a second side, wherein the nasal arch is located substantially in the center of the bottom;
a light shield, the light shield attached around the nasal arch on the back side; and,
two diffuser holders fixedly attached to the first and second sides of the nasal arch, wherein each diffuser holder has an interior cavity and an opening, wherein the opening is smaller than the interior cavity, wherein the interior cavity is adapted to receive diffuser material.

2. A mask comprising:
a front side;
a back side;
a top;
a bottom;
a left side;
a right side; and,
at least one diffuser holder, wherein the at least one diffuser holder has an interior cavity.

3. The mask of claim 2, wherein the mask further comprises an adjustable strap, wherein the strap is connected to the left and right sides.

4. The mask of claim 3, wherein the mask further comprises a nasal arch having a top, a first side, and a second side, wherein the nasal arch is located substantially in the center of the bottom.

5. The mask of claim 4, wherein the mask further comprises a light shield, the light shield attached around the nasal arch on the back side.

6. The mask of claim 5, wherein the mask further comprises eye pockets, wherein the eye pockets are located on the back side.

7. The mask of claim 6, wherein the at least one diffuser holder is at least two diffuser holders fixedly attached to the first and second sides of the nasal arch, wherein each diffuser holder has an opening, wherein the opening is smaller than the interior cavity, wherein the interior cavity is adapted to receive diffuser material.

8. The mask of claim 7, wherein the front side and the back side are made of layered fabric.

9. The mask of claim 8, wherein the layered fabric is silk or satin.

10. A method for using a sleep mask for aromatherapy, the method comprising the steps of:
providing a sleep mask comprising:
a front side;
a back side;
a top;
a bottom;
a left side;
a right side; and,
at least one diffuser holder, wherein the at least one diffuser holder has an interior cavity;
inserting diffuser material into the at least one diffuser holder; and,
applying the sleep mask to a user's head.

11. The method of claim 10, wherein the mask further comprises an adjustable strap, wherein the strap is connected to the left and right sides.

12. The method of claim 11, wherein the mask further comprises a nasal arch having a top, a first side, and a second side, wherein the nasal arch is located substantially in the center of the bottom.

13. The method of claim 12, wherein the mask further comprises a light shield, the light shield attached around the nasal arch on the back side.

14. The method of claim 13, wherein the mask further comprises eye pockets, wherein the eye pockets are located on the back side.

15. The method of claim 14, wherein the at least one diffuser holder is at least two diffuser holders fixedly attached to the first and second sides of the nasal arch, wherein each diffuser holder has an opening, wherein the opening is smaller than the interior cavity, wherein the interior cavity is adapted to receive diffuser material.

16. The method of claim 15, wherein the front side and the back side are made of layered fabric.

17. The method of claim 16, wherein the layered fabric is silk or satin.
